(19) United States
(12) Patent Application Publication
(10) Pub. No.: US 2014/0088560 A1
(43) Pub. Date: Mar. 27, 2014

(54) INTRA-LUMINAL ACCESS APPARATUS AND METHODS OF USING THE SAME

(71) Applicants: Robert J. Min, Pelham Manor, NY (US); Gary S. Dorfman, Saunderstown, RI (US)

(72) Inventors: Robert J. Min, Pelham Manor, NY (US); Gary S. Dorfman, Saunderstown, RI (US)

(73) Assignee: CORNELL UNIVERSITY, ITHACA, NY (US)

(21) Appl. No.: 14/008,762

(22) PCT Filed: Mar. 30, 2012
(86) PCT No.: PCT/US2012/031517

§ 371 Date: Dec. 10, 2013

Related U.S. Application Data
(60) Provisional application No. 61/469,470, filed on Mar. 30, 2011.

(51) Int. Cl. A61M 25/09; A61M 25/06

(52) U.S. Cl. CPC A61M 25/09; A61M 25/0668
USPC 604/510; 604/528

(57) ABSTRACT

Apparatus and methods providing intra-luminal access and, in particular, to needle, catheter, and guidewire structures and methods of using the same to access a vascular or other body tissue lumen.

### CROSS-REFERENCE TO RELATED APPLICATION

This application is related to and derives priority from U.S. Provisional Patent Application Ser. No. 61/469,470, filed 30 Mar. 2011 and titled “Intra-Luminal Access Apparatus and Methods of Using the Same,” the contents of which are incorporated herein fully by reference.

### TECHNICAL FIELD

Embodiments of the invention relate generally to apparatus and methods providing intra-luminal access and, in particular, to needle, catheter, and guidewire structures and methods of using the same to access a vascular or other body tissue lumen.

### BACKGROUND

Intra-luminal access, for example, access to the vasculature or into a hollow body cavity is accomplished by one of two general methods. First, there is the direct puncture method that involves accessing the lumen, e.g., a blood vessel, by use of a needle with or without an integral sheath. If there is no sheath all interventions (e.g., injection or administration of agents) are performed through the needle. If there is an integral sheath, the sheath is manipulated over the needle into the lumen and the needle is removed. In this variant of the direct puncture method all interventions are performed through the sheath. This method limits the length of any indwelling sheath to the length of the puncture needle and the internal diameter of any indwelling sheath to a diameter not significantly greater than the external diameter of the integral needle. Hence, larger and longer sheaths require the use of larger and longer needles. This relationship limits the length and diameter of permanent catheters for reasons of patient safety and operator convenience.

The second general method for intra-luminal access uses the Seldinger technique. This technique is based on the use of a guidewire to maintain lumen access, and all sheath and catheter placements and exchanges are dependent on that guidewire for lumen access. The initial lumen puncture is made utilizing a needle with or without an integral sheath. A guidewire is then placed via the needle or the sheath. The needle or integral sheath is then removed from the lumen over the guidewire; subsequently, dilators, larger sheaths, and/or catheters are placed over that same guidewire. If exchanges of catheters are necessary, all such exchanges are performed over a guidewire that is first placed through the indwelling catheter with removal of the indwelling catheter over the guidewire and subsequent placement of the new catheter(s) over that same guidewire. Since serial exchanges are possible with this technique, the diameter and length of indwelling catheters and sheaths is limited only by patient anatomy and/or functional/physiologic parameters of the vascular system.

The guidewires typically employed for such procedures employ a central metallic core with a wound wire (flat or round) surrounding the core. In some embodiments of guidewires, there may be no wound wire surrounding the core. In some cases the surrounding material is plastic rather than metallic and in some cases the entire guidewire or a portion of the guidewire is coated to decrease friction and diminish the tendency for blood to coagulate on the wire.

The Seldinger technique when used by properly trained professionals in controlled settings has been found to be safe and effective in clinical care. However, this technique has translated from the operating room, procedure room, and catheterization laboratory to inpatient care units and even the outpatient setting. It is used by mid-level providers with varying levels of training and experience, in addition to use by physicians. Specifically, it is used for the initial placement and subsequent exchanges of catheters for central venous access as well as for access to various hollow organs (gastrointestinal and genitourinary most predominantly). In these less controlled settings various adverse events and technical malfunctions occur with greater frequency (increased prevalence per 1000 patients and an increased number of patients treated by use of this technique both contribute to the increased frequency of adverse events and technical malfunctions). One malfunction that occurs not infrequently is loss of control of the guidewire during the initial placement of indwelling catheters or during the exchange of indwelling catheters. This malfunction most frequently necessitates a secondary procedure to retrieve the guidewire from the vascular lumen or the hollow organ so as to avoid long-term adverse consequences. It would be preferable to avoid this technical malfunction rather than to intervene after the fact.

If the guidewire were incapable of passing into the vascular or organ lumen through the puncture needle and/or the catheter, it would be impossible for the operator to lose control of the guidewire. In practice, interventional radiologists have been known to apply a surgical clamp to the external end of guidewires during procedures so as to avoid the possibility of losing control of the guidewire during long and complex procedures that involve substantial manipulations. However, this requires great care as the surgical clamp could damage the guidewire thereby rendering it useless for subsequent catheter exchanges and/or manipulations. Furthermore, placing a surgical clamp on the external end of the guidewire requires an “active intervention” on the part of the operator and if this step is inadvertently not performed, the required margin of safety is lost.

Indwelling catheters are most often constructed of soft, relatively atraumatic materials in order to decrease the rate of adverse events related to the interaction of the catheter with the vascular endothelium. These soft indwelling catheters generally do not have sufficient stiffness to be used for dilating the puncture site or to be delivered directly after needle puncture. In addition, the soft materials that are used in the construction of these indwelling catheters frequently have higher resistance during insertion through the skin tract (i.e., these catheters have greater friction during placement). Therefore, indwelling catheters are frequently placed through large bore sheaths and only after dilation of the puncture site with stiff dilators and or a stiff dilator on which the sheath is delivered into the lumen with subsequent placement of the indwelling catheter through the sheath. Each of these serial maneuvers provides an opportunity for loss of control of the guidewire or potentially inadvertent loss of access if the guidewire is dislodged from the lumen. Therefore there would be an advantage if the indwelling catheter could have increased stiffness and lubricity during placement, but could then become soft and atraumatic once within the lumen.

Employment of methods and devices to secure guidewires and to place catheters may require additional new structures. Various embodiments of structures, devices and methods are described herein to provide intra-luminal access.

### SUMMARY

An embodiment of the invention is a guide wire. The guidewire includes an elongate body portion having a proximal end (defined as the end that would normally remain exterior to or outside of the body) and a distal end (defined as the end that would normally be inside the body lumen into which access has been achieved), and an impediment structure disposed on the guidewire at or adjacent the proximal end. In various non-limiting aspects, the guidewire may have the following characteristics or features:

wherein the proximal end has a dimension d2 and the impediment structure has a dimension d1 that is larger than d2;

wherein the impediment structure comprises an enlarged diameter portion of the proximal end
wherein the impediment structure comprises a coiled portion of the body portion;
wherein the impediment structure comprises a deformed portion of the body portion;

wherein the impediment structure is removeably disposed on the proximal end;

wherein the impediment structure is permanently secured to the body portion;
wherein the impediment structure is frictionally secured with the body portion;
wherein the impediment structure is threaded onto the body portion;

wherein the impediment structure is bonded to the body portion;

wherein the impediment structure is deformable and is characterized by an impeding configuration and a release configuration.
further comprising a member to maintain the impediment structure in the release configuration.
   wherein the impediment structure has a foam or a gel construction;

wherein the impediment structure has at least one collapsible wing member radially extending from the body portion;

wherein the impediment structure is a material coating on the body portion;

wherein the impediment structure comprises at least one of a knurl, a scallop, and a ridge structure formed in the body portion.

An embodiment of the invention is a needle. The needle includes at least a first portion and a second portion separably connected to form an elongate lumen from a proximal end to a distal end thereof. However, based on superficial appearance and manufacturing process, the first and second portions of the needle may not be discernable as discrete structures. In various non-limiting aspects the needle may have the following characteristics or features:

wherein the first portion and the second portion are integral and are separable along a joining interface comprising a scored or localized area of weakness to allow the first portion to be separated from the second portion;

further comprising a grasping member formed on each of the first portion and the second portion to facilitate separation;
wherein the grasping member encircles a distal end of the needle;

further comprising a removable stylet disposed within the lumen.

An embodiment of the invention is a catheter. The catheter includes a soft, atraumatic lumen portion, and a reinforcing exoskeleton joined with the lumen portion. In various non-limiting aspects the guidewire may have the following characteristics or features:

wherein the reinforcing exoskeleton is disposed on an exterior surface of the lumen;
wherein the reinforcing exoskeleton is disposed within a wall of the lumen;
wherein the reinforcing exoskeleton is characterized by a first state wherein it has a first state rigidity substantially greater than a rigidity of the lumen and a second state wherein it has a second state rigidity equal to or less than the rigidity of the lumen;
   wherein the reinforcing exoskeleton is characterized by a transition temperature below which it has the first state rigidity and above which it has the second state rigidity;
   wherein the reinforcing exoskeleton is responsive to an external stimulus to transition from the first state rigidity to the second state rigidity;
      wherein the external stimulus is at least one of heat, electricity, magnetism, and ultrasound;
   wherein the reinforcing exoskeleton is bioabsorbable or biodegradable;
   wherein the reinforcing exoskeleton comprises a web-like structure;
   wherein the reinforcing exoskeleton comprises a sheath;
      wherein the sheath is disposed on an exterior surface of the lumen;
      wherein the sheath is separable into at least a first portion and a second portion upon placement of the lumen within a vascular or organ lumen;
         wherein the sheath is separable from a proximal end to a distal end thereof;
      wherein the sheath may be removed while leaving the catheter in place within the body lumen once the sheath has been separated into at least a first portion and a second portion.

An embodiment of the invention is the use of a guidewire for providing intra-luminal access.

An embodiment of the invention is the use of a guidewire for placing or replacing an indwelling catheter.

An embodiment of the invention is the use of a guidewire for placing or replacing a catheter.

An embodiment of the invention is the use of a guidewire for providing intra-luminal access.

An embodiment of the invention is a needle assembly. The needle assembly includes a guidewire including a docking structure disposed on a portion thereof, and a needle having an elongate cannulated portion and including a compatible docking structure disposed at a proximal end of the cannulated portion, wherein the cannulated portion is removeably connectable to the guidewire via the docking structure and the compatible docking structure. In various non-limiting aspects the guidewire may have the following characteristics or features:

further comprising a hub disposed at the proximal end of the cannulated portion;
wherein the hub is removable;
wherein the hub is splitable;

wherein the docking structure has a male configuration and the compatible docking structure has a female configuration;

wherein the docking structure is disposed at or adjacent a distal end of the guidewire;
wherein the guidewire further comprises an impediment structure;

wherein the cannulated portion is substantially rigid;

wherein the docking structure is tapered at a non-docking end thereof;

wherein the cannulated portion has a blunted proximal end so as to prevent inadvertent trauma to the guidewire that has been docked to the cannulated portion of the needle.

### BRIEF DESCRIPTION OF THE DRAWINGS

FIG. 1 is a graphic depiction of a guidewire including an impediment end structure in accordance with one or more of the herein described embodiments;

FIG. 2a is an enlarged view of a guidewire end showing an impediment end structure in an impeding configuration in accordance with an aspect of the invention; FIG. 2b is an enlarged view of the impediment end structure shown in FIG. 2a in a release configuration, according to an aspect of the invention;

FIG. 3 is a schematic depiction of an impediment end structure incorporating a disengagement alert device, according to an aspect of the invention;

FIG. 4a is an enlarged view of a guidewire end showing an impediment end structure in an impeding configuration, in accordance with an embodiment described herein; FIG. 4b is an enlarged view of the impediment end structure shown in FIG. 4a in a release configuration, according to an aspect of the invention;

FIG. 5a is an enlarged view of a guidewire end showing an impediment end structure in an impeding configuration, in accordance with an embodiment described herein; FIG. 5b is an enlarged view of the impediment end structure shown in FIG. 5a in a release configuration, according to an aspect of the invention;

FIG. 6a is an enlarged view of a guidewire end showing an impediment end structure in an impeding configuration, in accordance with an embodiment described herein; FIG. 6b is an enlarged view of the impediment end structure shown in FIG. 6a in a release configuration, according to an aspect of the invention; FIG. 6c is an enlarged view of the impediment end structure shown in FIG. 4a in a release configuration secured by a sleeve, according to an aspect of the invention;

FIG. 7a is an enlarged view of a guidewire end showing an impediment end structure in an impeding configuration, in accordance with an embodiment described herein; FIG. 7b is an enlarged view of the impediment end structure shown in FIG. 3a in a release configuration, according to aspects of the invention;

FIG. 8a is an enlarged view of a guidewire end showing an impediment end structure in an impeding configuration, in accordance with an embodiment described herein; FIG. 8b is an enlarged view of the impediment end structure shown in FIG. 8a in a release configuration, according to an aspect of the invention;

FIG. 9 is an enlarged view of a guidewire end showing an impediment end structure in accordance with an embodiment described herein;

FIG. 10 is an enlarged view of a guidewire end showing an impediment end structure in accordance with an embodiment described herein;

FIG. 11a is a graphic depiction of a guidewire and needle incorporating a docking structure; FIG. 11b is a graphic depiction of the guidewire and needle shown in FIG. 11a with the needle docked to the guidewire, according to aspects of the invention;

FIG. 12a is a graphic depiction of a needle in accordance with an embodiment described herein; FIG. 12b is a graphic depiction of the needle of FIG. 12a in a parted state; FIG. 12c is a graphic depiction of an end of the needle of FIG. 12a showing grasping wings for in-situ parting of the needle into needle portions;

FIG. 13 is a graphic depiction of a catheter in accordance with an embodiment described herein;

FIG. 14 is a graphic depiction of a catheter in accordance with an embodiment described herein;

FIG. 15 is a graphic depiction of a catheter in accordance with an embodiment described herein; and

FIG. 16 is a graphic depiction of the catheter of FIG. 15 during a placement procedure, according to an illustrative aspect of the invention.

### DETAILED DESCRIPTION OF EXEMPLARY EMBODIMENTS OF THE INVENTION

Embodiments and aspects of the invention pertain to various apparatus and methods of using such apparatus for intra-luminal access. In general, intra-luminal access refers to access to a vascular lumen, hollow organ, or other body cavity via needle puncture, Seldinger technique, or similar methods. As used herein, a lumen or an accessed lumen may refer to a vascular lumen, hollow organ, or virtually any patent structure of the body.

### Guidewire

In accordance with several of the herein described embodiments, an apparatus, e.g., in the form of a guidewire is formed to have an impediment structure disposed on or adjacent (near) the external or proximal end of the guidewire. The impediment structure is designed to prevent entry of the external end of the guidewire into the needle, sheath, catheter, and/or other structure providing access to a lumen and, therefore, impedes entry of the guidewire into the lumen itself. The impediment structure may be permanent (i.e., impassable) or may be overcome (i.e., passed-over) by active intervention, wherein the impediment structure may have an impeding configuration and a release configuration, the release configuration being realized only upon active intervention by the operator.

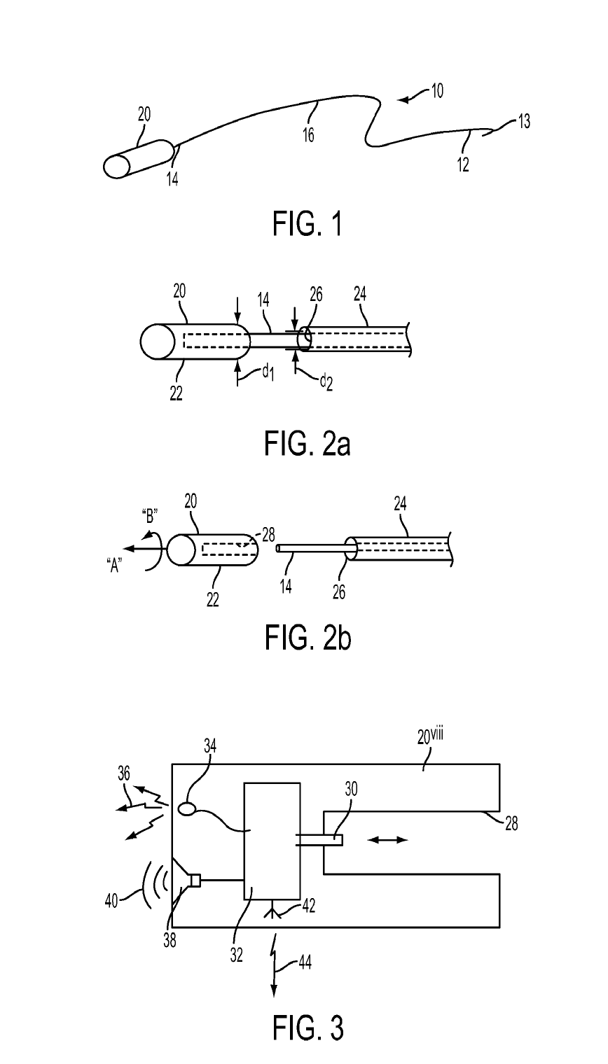

Referring to FIG. 1, a guidewire 10 includes a body portion 16 having a distal or ‘insertable’ end 12 and an external or proximal end 14. The configuration of the guidewire 10 body portion 16 from the external end 14 to the internal end 12 may be of any typical construction. Except as expressly noted and described herein, practice of the herein described inventions does not depend on the actual physical construction of the guidewire 10, which therefore does not form part of the invention. As such, the guide wire 10 may have a single element, wound core element, uncoated, coated or any commercially available or later developed construction. The distal end 12, shown in FIG. 1 with a floppy “J” configuration 13 may alternatively have a soft straight end, stiff straight end, steerable end, malleable end, tapered end, or any commercially available or later developed guidewire end construction. Proximal end 14 includes an impediment structure 20.

The impediment structure 20 may be permanent (i.e., having a size, shape, and/or configuration that will not allow a needle, sheath, dilator, or catheter to pass-over). Alternatively, it may be configured to be overcome (passed-over) by active intervention of the operator. The impediment structure 20 in an impeding configuration prevents passage of the impediment structure and associated portion of the guidewire through a needle, sheath, dilator, catheter, and/or vascular lumen access device or puncture site, collectively referred to herein as lumen. The impediment structure 20 may be metallic, plastic, compressible foam or gel, or another material suitable for medical applications. The presence of the impediment structure 20, particularly if permanently affixed to the guidewire 10, mandates that the catheter or other device intended for placement over the guidewire be pre-positioned onto the guidewire; i.e., prior to its insertion into the lumen. Additionally, since the guidewire 10 must first be placed through the puncture needle, a hitherto unavailable in-situ partable needle would be advantageous, an embodiment of which is described below.

FIG. 2a shows an enlarged view of the external end region 14 of the guidewire 10 and the impediment structure 20 and, additionally, a portion of a sheath 24 positioned over a portion of the guidewire. The impediment structure 20 is depicted as substantially cylindrical and can be affixed either permanently or removeably to the external end 14 of the guidewire 10. The impediment structure 20 may have virtually any cross-sectional configuration; e.g., cylindrical as shown in FIG. 1 or square or rectangular, triangular, convex, concave, irregular, or the like. Moreover, although an outer surface 22 of the impediment structure 20 appears smooth, as depicted in FIG. 1, the same can be made knurled, textured, or otherwise formed to facilitate gripping of the impediment structure 20. Other portions of the impediment structure 20 can also be formed to facilitate gripping or grasping of the same by an operator during use of the guidewire 10.

The impediment structure 20 may have a substantially uniform diameter, depicted as diameter d1 in FIG. 2a, or may alternatively be formed to have a portion of at least diameter d1 while other portions have a different (e.g., smaller) diameter. Additionally, the diameter d1 of the impediment structure 20 does not require the diameter profile to be circular. In view of the impediment structure 20 having various configurations, the impediment structure 20 may have a width or thickness of d1, an elliptical major diameter of d1, and the like. The impediment structure 20 diameter d1 is chosen so as to be larger than the internal diameter d2 of the lumen 26 as depicted in FIG. 2a, where the lumen 26 is the lumen defined by the sheath 24. In this manner, the end 14 of the guidewire 10 is prevented from entering the lumen 26 because the impediment structure 20 has at least a portion with a diameter d1 greater than the internal diameter d2 of the lumen 26. In alternative terms, the impediment structure 20 may have an area in cross-section greater than an area in cross-section of the lumen 26 such that the impediment structure 20 cannot enter the lumen 26. Therefore, as depicted in FIG. 2a, the impediment structure 20 is in an impeding configuration.

As noted, the impediment structure 20 may be permanently affixed to, or adjacent, the end 14 of the guidewire 10. In such instances, the sheath 24 would be disposed or pre-positioned about the guidewire 10 during placement of the guidewire 10 via a needle or other suitable method of introducing the guidewire and sheath into a lumen. Provision would then be required to remove the introducing needle or other structure, and suitable embodiments are described below.

Alternatively, or as shown in FIG. 2b, the impediment structure 20 may be removable from the end region 14 of the guidewire 10 to provide a release configuration. Removal of the impediment structure 20 from the guidewire 10 is at least one possible way in which the impediment structure 20 may be overcome by active intervention to facilitate such activities as needle removal, sheath placement and/or catheter placement or replacement, for example. The impediment structure 20 may be fitted to the end 14 by friction or otherwise. As depicted in FIG. 2b, the impediment structure 20 is formed with a blind bore 28 into which the end 14 is fitted and retained by friction between the end 14 and the bore 28. To enhance frictional engagement, although not depicted, the end 14 may have a conical or tapered shape fitting into a corresponding conical or tapered bore formed in the impediment. Still further, the end 14 may be formed with threads and the bore 28 formed with corresponding threads, the impediment structure therefore being retained to the guidewire 10, removeably, by mutual engagement of the threads. The end 14 and the bore 28 may alternatively be formed with a quarter-turn or similar locking structure that facilitates quickly locking and unlocking the impediment structure 20 to the guidewire 10. Similar and alternative structures for mechanically locking the impediment structure 20 the guidewire 10 may be used. The impediment structure 20 may therefore be overcome by active intervention such as applying a force along the arrow “A” or a rotational force along the arrow “B,” or a combination thereof.

Once the operator removes the impediment structure 20, it may be desirable to provide a visual and/or tactile and/or aural reminder that the impediment structure has been removed. In this way, a persistent and obvious reminder is activated and can only be disabled by re-applying the impediment structure 20 to the appropriate location on the end 14 of the guidewire 10.

Referring to FIG. 3, the impediment structure 20^{viii} includes an alert device 32. As depicted in FIG. 3, a switch 30 is disposed at the end of bore 28 that is engaged or disengaged by the guidewire when the impediment structure 20^{viii} is disposed on the guidewire and removed from the guidewire, respectively. When the impediment structure 20^{viii} is disposed on a guidewire, the guidewire engages the switch 30 providing a signal to the device 32 based upon the position of the switch 30. The device 32 may have a self-contained power source, such as a battery and circuitry to detect the position of the switch 30 and the associated signal. When the impediment structure 20^{viii} is on the guidewire, the device 32 is active, but in a standby mode. When the impediment structure 20^{viii} is removed from the guidewire, the device 32 detects this based upon the position of the switch 30 and activates any one or more of various indication by causing LED 34 to emit light 36, speaker 38 to emit sound 40, or antenna 42 to emit radio signals 44 that are picked up by an in-theatre receiver to provide a theatre-wide alert. The device 32 may also include a vibrator device causing the impediment structure to vibrate once removed from the guidewire or similarly to provide a tactile signal to the operator.

A passive reminder that the impediment structure 20^{viii} has been removed from the guidewire may include coloring the end of the guidewire with an alerting color such as safety orange, greens, yellows, and the like.

Overcoming the impediment structure 20 by active intervention facilitates common procedures with which the guidewire 10 may be used, such as placing or replacing catheters and the like. In alternative embodiments described herein below, the impediment structure 20 may be overcome by active intervention without actually removing it from the guidewire 10, and such embodiments have additional advantages.

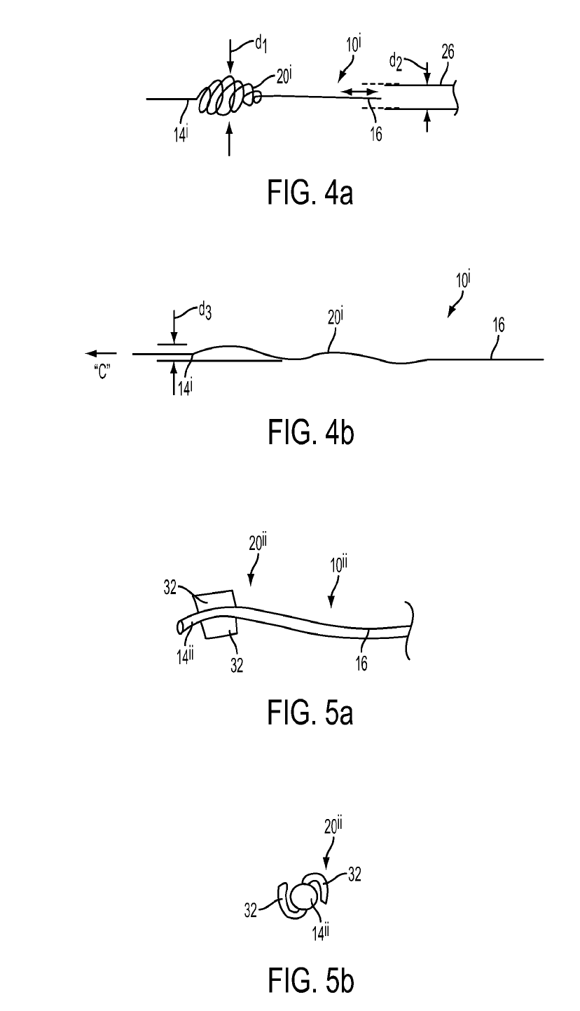

FIGS. 4a and 4b illustrate an alternative impediment structure design that may be permanently affixed to, or adjacent, the end 14ⁱ of a guidewire 10ⁱ, which is deformable or alterable in such a way as to allow forced passage of a lumen (e.g., dilators, sheaths, and/or catheters) (not shown) over the impediment structure 20ⁱ to provide a release configuration. Passage of the lumen 26 over the impediment structure 20ⁱ, however, is only possible with a conscious effort on the part of the operator to actively intervene and overcome the impediment structure 20ⁱ. This would prevent the unintended loss of control of the guidewire 10ⁱ as the effort required to deform or alter the impediment structure 20ⁱ would be impossible or highly unlikely to be applied without intent. Consistent with a primary object of an embodiment of the invention, this impediment design aspect and any other continues to provide a deterrent to the entry of (and possible loss of) the external end of the guidewire into a lumen.

Referring still to FIGS. 4a and 4b, one example of a deformable impediment structure 20ⁱ formed integral with the guidewire 10ⁱ is a portion of the guidewire 10ⁱ itself, or a section of a surrounding or wound wire in a wound wire-type guidewire, that is redundant or more loosely wound forming a coil at or near the external end 14ⁱ. In this manner, the impediment structure 20ⁱ has a normal (i.e., unbiased or unstretched) configuration characterized by a cross sectional dimension or width portion of d1, greater than the inner diameter d2 of a lumen 26. However, by biasing (tensioning or applying a force along the arrow “C” depicted in FIG. 4b to the guidewire 10ⁱ from either side of the impediment structure 20ⁱ) the guidewire, it contracts to a release configuration that will allow entry into the lumen. For example, the wound wire stretches onto the guidewire core thereby decreasing its diameter. The decrease in diameter of the impediment structure 20ⁱ allows the lumen to be withdrawn over the impediment structure 20ⁱ and off of the wire, and a new lumen, e.g., a new indwelling catheter, to be placed onto the guidewire 10ⁱ for subsequent insertion into the vascular or organ lumen. In a wound wire-type guidewire, the impediment structure 20ⁱ would be facilitated by the external or proximal end 14ⁱ of the guidewire 10ⁱ from just adjacent the impediment (and the puncture site into the body lumen) to the impediment structure 20ⁱ through to the proximal end 14ⁱ being constructed such that the core is not attached to the winding. Additionally, the core may be coated so as to decrease the sliding friction between the core and the winding.

In another alternative embodiment of a guidewire, guidewire 10ⁱⁱ includes an impediment structure 20ⁱⁱ as illustrated in FIGS. 5a and 5b. As depicted, the impediment structure 20ⁱⁱ includes one or multiple extensions (e.g., flaps or wing)s 32 that protrude radially outwardly from the end 14ⁱⁱ. With the wings 32 projecting outwardly from the end 14ⁱⁱ (FIG. 5a), the guidewire 10ⁱⁱ cannot pass into the lumen. By active intervention, the wings 32 can be folded and compressed against the end 14ⁱⁱ as shown in FIG. 5b to allow passage of the lumen over the compressed impediment structure.

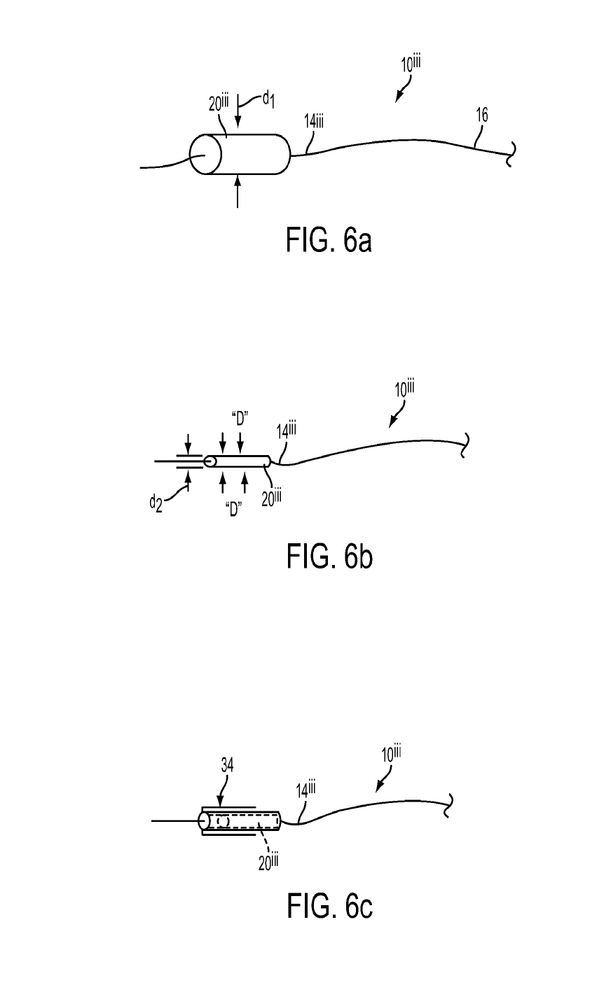

In still another embodiment of a guidewire as illustrated in FIGS. 6a and 6b, guidewire 10ⁱⁱⁱ includes an impediment structure 20ⁱⁱⁱ. As depicted, the impediment structure 20ⁱⁱⁱ is a deformable foam or gel structure disposed on the end 14ⁱⁱⁱ. In a relaxed state, shown in FIG. 6a, the impediment structure 20ⁱⁱⁱ has a portion in cross-section or a diameter that is too large to pass into the lumen (not shown). Active intervention by compressing the impediment structure 20ⁱⁱⁱ along the end 14ⁱⁱⁱ by application of force along arrows “D” as shown in FIG. 6b reduces the diameter of the impediment structure 20ⁱⁱⁱ to a smaller diameter d2 and allows the lumen (not shown) to pass over the impediment structure 20ⁱⁱⁱ. The foam or gel material may be selected to have a slow recovery so that once compressed it remains so for a time period sufficient to easily manipulate the lumen over the same.

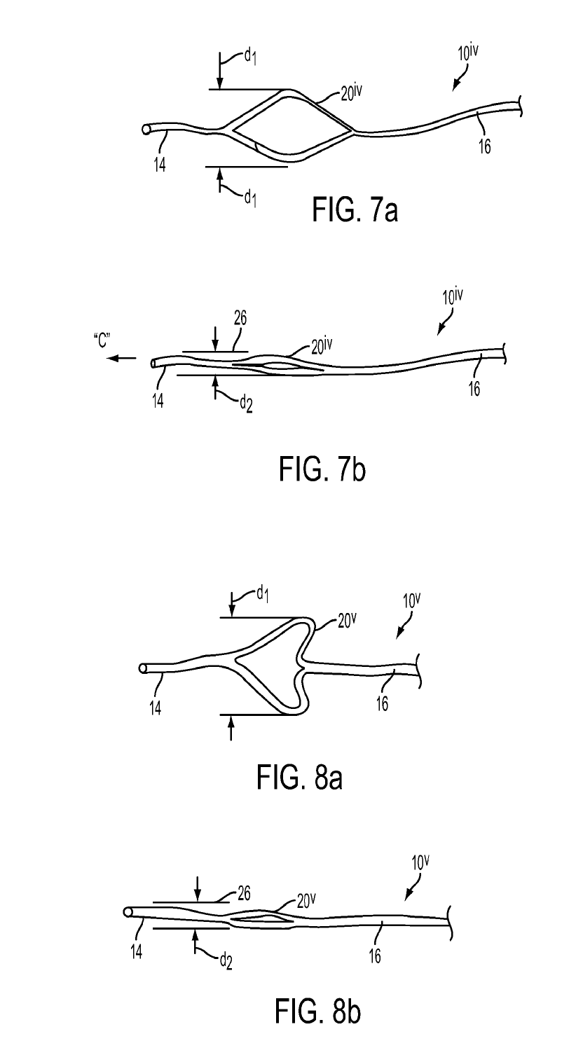

In connection with the described embodiments of impediment structures 20, and especially in connection with those embodiments depicted in FIGS. 5a/5b and 6a/6b and, which will be described in connection with FIGS. 7a/7b and 8a/8b, there could be a sleeve 34, depicted in FIG. 6c, that when placed over the impediment structure 20ⁱⁱⁱ maintains the compression of the impediment structure 20ⁱⁱⁱ to allow passage of lumen over the impediment structure 20ⁱⁱⁱ. When the sleeve 34 is in its “resting” position, i.e., not covering the impediment structure 20ⁱⁱⁱ the impediment structure 20ⁱⁱⁱ does not allow passage of the lumen over the impediment structure 20ⁱⁱⁱ or entry of the guidewire end 14ⁱⁱⁱ into the lumen. An outer diameter of the sleeve 34 is sized to allow over passage of the lumen 26. Other deformable-type impediment structures may be achieved by applying or using metallic, plastic, fabric, or other materials with or without an assisting sleeve 34.

FIGS. 7a and 7b illustrate an example of a deformable impediment structure 20^{iv} formed integral with the guidewire 10^{iv}, where the impediment structure 20^{iv} is a portion of the guidewire 10^{iv} itself or a section of a surrounding, or wound wire in a wound wire-type guidewire that is redundant or more loosely wound, forming a diamond, parallelogram, or similar structure at or adjacent the external end 14^{iv}. In this manner, the impediment structure 20^{iv} has a normal configuration characterized by a dimension or width portion of d1, greater than the inner diameter d2 of the lumen 26. However, by “tensioning”, e.g., by applying a force along the arrow “C” depicted in FIG. 7b to the guidewire 10^{iv} from either side of the impediment structure 20^{iv}, it contracts to a release configuration. For example, the diamond structure collapses onto itself or the guidewire core thereby decreasing its diameter. The decrease in diameter of the impediment structure 20^{iv} allows the lumen to be withdrawn over the impediment structure 20^{iv} and off of the wire, and a new lumen, e.g., a new indwelling catheter, to be placed onto the guidewire 10^{iv} for subsequent insertion into the vascular or organ lumen. In a wound wire-type guidewire, the impediment structure 20^{iv} would be facilitated by the external or proximal end 14^{iv} of the guidewire 10^{iv} from just adjacent the impediment structure (and the puncture site into the body lumen) to the impediment structure 20^{iv} through to the end 14^{iv} being constructed such that the core is not attached to the winding. Additionally, the core may be coated so as to decrease the sliding friction between the core and the winding.

The diamond shape of the impediment structure 20^{iv} may also allow or facilitate over-passage of the lumen 26 by collapsing within the lumen 26 as the operator applies a sufficiently large relative force between the lumen 26 and the guidewire 10^{iv}. The force required would be sufficiently great so that the guidewire 10^{iv} could not on its own pass through the lumen 26.

Referring to FIGS. 8a and 8b, another example of a deformable impediment structure 20^{v} formed integral with the guidewire 10^{v} is a portion of the guidewire 10^{v} itself or a section of a surrounding or wound wire in a wound wire-type guidewire that is redundant or more loosely wound forming a triangular, parallelogram, or similar structure at or adjacent the external end 14^{v}. In this manner, the impediment structure 20^{v} has a normal configuration characterized by a dimension or width portion of d1, greater than the inner diameter d2 of the lumen 26. However, by “tensioning”, e.g., by applying a force along the guidewire 10^{v} from either side of the impediment structure 20^{v}, it contracts to a release configuration. For example, the triangular structure collapses onto itself or the guidewire core thereby decreasing its diameter. The decrease in diameter of the impediment structure 20^{v} allows the lumen to be withdrawn over the impediment structure 20^{v} and off of the wire and a new lumen, e.g., a new indwelling catheter, to be placed onto the guidewire 10^{v} for subsequent insertion into the vascular or organ lumen. In a wound wire-type guidewire, the impediment structure 20^{v} would be facilitated by the external or proximal end 14^{v} of the guidewire 10^{v} from just adjacent the impediment structure (and the puncture site into the body lumen) to the impediment structure 20^{v} through to the end 14^{v} being constructed such that the core is not attached to the winding. Additionally, the core may be coated so as to decrease the sliding friction between the core and the winding.

The triangular shape of the impediment structure 20^{v} may also allow or facilitate passage of the lumen 26 over and onto the guidewire 10^{v} by collapsing within the lumen 26 as the operator applies a sufficiently large relative force between the lumen 26 and the guidewire 10^{v}. However, the base leg of the triangular shape would prevent such collapsing of the triangular shaped impediment structure 20^{v} preventing the guidewire from entering the lumen 26 except with operator intervention to sufficiently collapse the impediment structure 20^{v}.

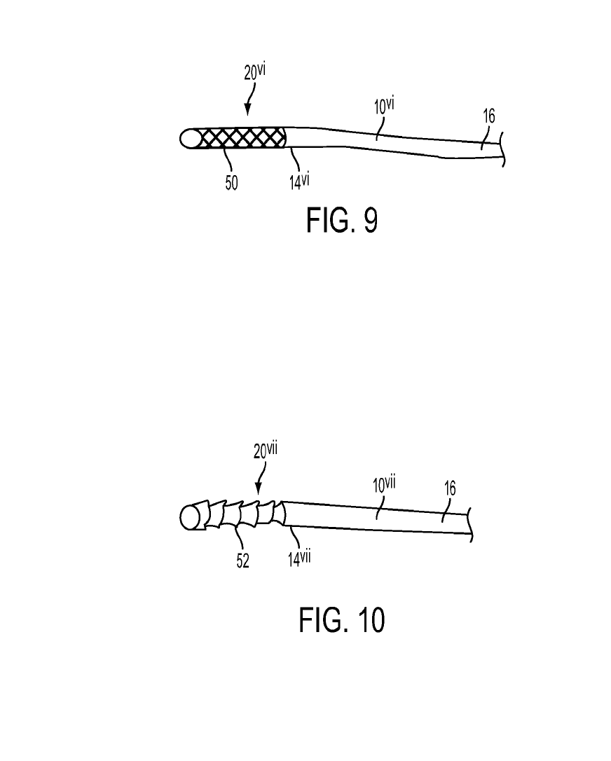

FIGS. 9 and 10 illustrate still additional examples of effective impediment structures that may be formed on the guidewire itself. In FIG. 9, the impediment structure 20^{vi} may be formed by applying a high friction material 50 such as a tacky polymer or similar coating to the end 14^{vi} of the guidewire 10^{vi}. The high friction of the material 38 would inhibit passage of the needle and/or catheter over the end 14^{vi}. The high friction of the material 38 may be overcome and made more lubricious by applying a biocompatible resistance reducing material (not depicted) that is easily removable or naturally short-lived. Hence, without the resistance reducing material applied, the impediment structure 20^{vi} presents an impediment to normal operator effort, but with application of the lubricant the impediment structure 20^{vi} is easily overcome by normal operator effort to allow the lumen 26 to pass over the impediment structure 20^{vi}. Once the resistance reducing material is removed, evaporated, or otherwise made inactive, the impediment structure 20^{vi} would once more present an impediment to passage of the lumen 26 using only normal operator effort.

As depicted in FIG. 10, an impediment structure 20^{vii} may be provided at or near the end 14^{vii} of the guidewire 10^{vii}, such as providing a knurled, scalloped, ridged or the like surface 40 to increase the effort required to pass the needle and/or catheter over the end 14^{vii}. The impediment structure 20^{vii} can be overcome similar to impediment structure 20^{vi} by application of user force, with or without lubricant.

### Dockable Needle

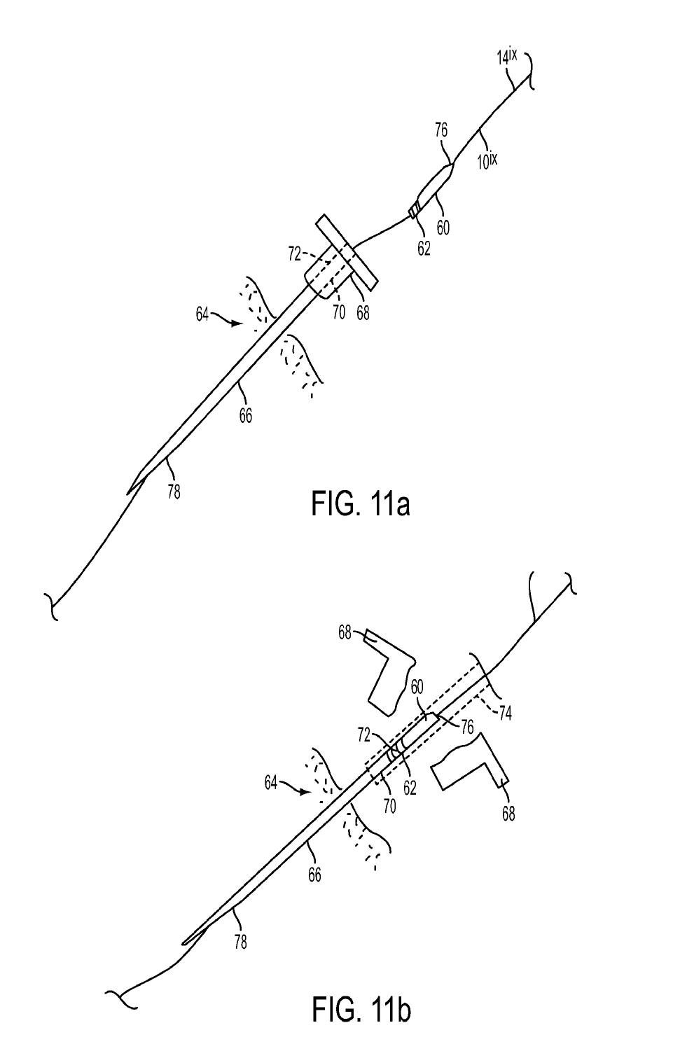

Referring to FIGS. 11a and 11b, a secure guidewire, e.g., guidewire 10^{ix} may be formed with an impediment structure in accordance with any of the herein described embodiments, but inclusion of an impediment structure is not required in connection with this embodiment. If an impediment structure is provided, the guidewire 10^{ix} may be pre-loaded with a catheter or other structure to be placed using the guidewire 10^{ix}.

Formed along the length of the guidewire 10^{ix} or at the end 14^{ix} is a docking structure 60. For example, the docking structure 60 may be formed on the guidewire 10^{ix} between the proximal end (the end that is eventually situated within the body lumen) and an impediment structure, if included, of the guidewire 10^{ix}. The docking structure 60 includes a male docking portion 62.

A needle 64 includes a cannulated portion 66 and a hub portion 68 at an entry end thereof. While the needle 64 may have a typical, substantially rigid construction, in an alternative form the cannulated portion 66 may have greater flexibility and less rigidity than typical needle constructions. In such embodiments of the needle 64, initial puncture and placement of the needle 64 may be assisted by a rigid stylet or trocar. The needle 64 may be formed from surgical metals but may as well be formed from suitable plastics, polymers, carbon, and the like and combinations thereof.

The hub 68 may further assist placement of the needle 64 and may be removable from the cannulated portion 66. The hub 68 may be removed from the cannulated portion 66 prior to placement of the guidewire 10^{ix}. In one aspect, the hub 68 is removed from the cannulated portion 66 after placement of the guidewire 10ᵢₓ, and then can be split or otherwise deconstructed to allow removal of the hub 68 from about the guidewire 10ᵢₓ, as depicted in FIG. 11b.

The cannulated portion 66 proximal end 70 is formed with a docking structure 72 that accepts the complimentary docking structure 62 of the guidewire. The docking structure 72 may be female and the docking structure 62 may be male, as shown in FIG. 11, or docking structure 72 may be male and the docking structure 62 may be female, or they may abut. The cannulated portion 66 may be secured to, i.e., docked, to docking structure 60 by engagement of the docking portion 72 with the docking portion 62. The docking portions 62 and 72 may be threaded for mutual engagement or otherwise formed to allow secure docking of the cannulated portion 66 with the docking structure 60, e.g., by friction fit or other methods known in the art.

Once docked to the guidewire 10^{ix}, the needle 64 and the guidewire 10^{ix} become a singular unit and are used to place catheters or other structures; for example, a catheter 74 is depicted in phantom in FIG. 11b being placed over the joined docking structure 60/needle 64 combination. An end 76 of the docking structure 60 may be tapered to facilitate passing of the catheter over the docking structure 60 and cannulated portion 66. After placement of the catheter or completion of the procedure, the guidewire 10^{ix} and the needle 64 are removed together.

As noted, the cannulated portion 66 may be flexible so as to be atraumatic when placed within a lumen and to facilitate further procedures. Additionally, the distal end 78 of the needle 64 may be formed to reduce the possibility of damaging or severing the guidewire 10ᵢₓ. Both the blunted end 78 and flexibility of the cannulated portion suggest the need to utilize a stylet or trocar for initial puncture and placement of the needle 64. The inner stylet may be either concentrically or eccentrically beveled to facilitate passage through the subcutaneous and muscular tissue as well as puncture of the vessel wall to gain access into the lumen. The stylet could be solid or hollow. If hollow, the stylet would allow the operator to recognize puncture of the vascular lumen through observation of blood return. Once access to the lumen has been achieved the stylet is removed.

### Partable Needle

Where the impediment structure is permanent to the guidewire external end, and not easily overcome by application of resistance reducing material or the like, provision must be made for withdrawing the needle. One possibility permitting removal of the needle is the use of an in-situ partable needle that may be parted into separate components and removed from about the guidewire without withdrawing the guidewire. Impediment structures that are deformable or provided with a means to be overcome by active intervention might be preferred for exchanges of previously placed indwelling catheters for new indwelling catheters. Of course, any of the embodiments can be used for newly placed catheters whether those catheters are meant for short-term or longer-term use.

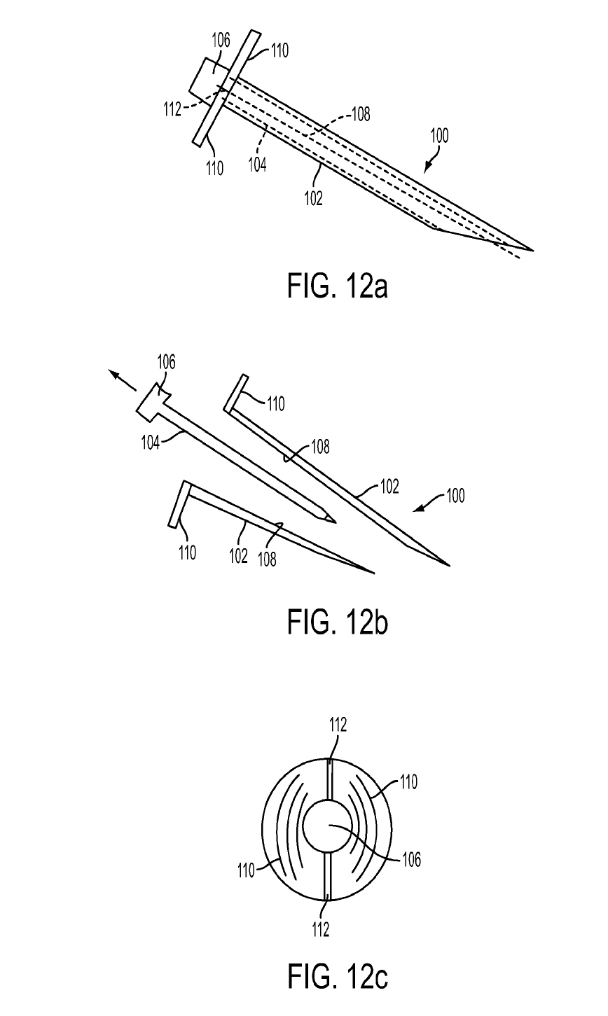

FIGS. 12a, 12b and 12c depict an embodiment of an in-situ partable needle 100. While peel-away sheaths have been described and are commercially available, most of these have almost no structural rigidity and require placement over a guidewire using the Seldinger technique as previously described in the background section. The reason for this is that such peel-away sheaths typically employ thin walls and must therefore be placed using an integral dilator that is then removed over the guidewire leaving only the peel-away sheath in place through which permanent or temporary catheters may be placed over the guidewire but through the sheath. Subsequently, the sheath is peeled away leaving only the catheter in place. However, control of the guidewire remains important and may not be maintained during these manipulations without use of an impediment structure formed to the guidewire.

The partable needle 100 would allow placement of a guidewire constructed in accordance with any of the foregoing embodiments through the needle 100 with the catheter pre-loaded thereon. The partable needle 100 could then be removed by separating (parting) the needle 100 along its length into two or more portions, rather than by removing it over the guidewire, which would be impossible as both the pre-loaded catheter and the impediment structure would preclude removal of the needle in the traditional method as described by, for example, the Seldinger technique.

In addition, the partable needle 100 would have utility for other purposes. For example, the size of the vascular puncture could be smaller using a partable needle 100 as compared with a needle carrying an integral sheath (e.g., so called “micropuncture” sets). This would allow the use of yet thinner and smaller catheters while still minimizing bleeding at the puncture site.

The partable needle 100 could be made from metal, plastics/(co)polymers, carbon, or other materials. In this manner, the needle 100 has an elongate lumen portion 102. The capacity to separate the needle 100 into two or more portions could be enhanced by providing notches at its distal end, scoring the needle along its length (depicted by its score line 108) or in a gentle spiral pattern and by adding grasping wings 110. Needle 100 strength and integrity could be maintained by an optional inner stylet 104 that would be removed after puncture by grasping and pulling an end portion 106 thereof. The stylet 104 may have a lumen to allow assessment of blood return during the puncture. The partable needle 100 could have a concentric tip or could be beveled, as depicted in the FIGS. 11a and 11b.

The wings 110 may be made continuous with a score portion or area of weakness 112 allowing them to be separated into separate wings for parting the needle 100 into portions. The wings 110 may be formed as separate members that snap or lock together. This structure of wings 110, either as a scored single piece or as joined multiple pieces provides rigidity to the distal end of the needle 100 and furthermore reduces the likelihood the needle 100 is inadvertently parted into portions.

The partable needle 100 is compatible with and therefore may be formed also to include a hub such as the hub 68 (FIG. 11b) to allow securing to a docking impediment structure disposed on a guidewire.

### Exoskeleton Catheter

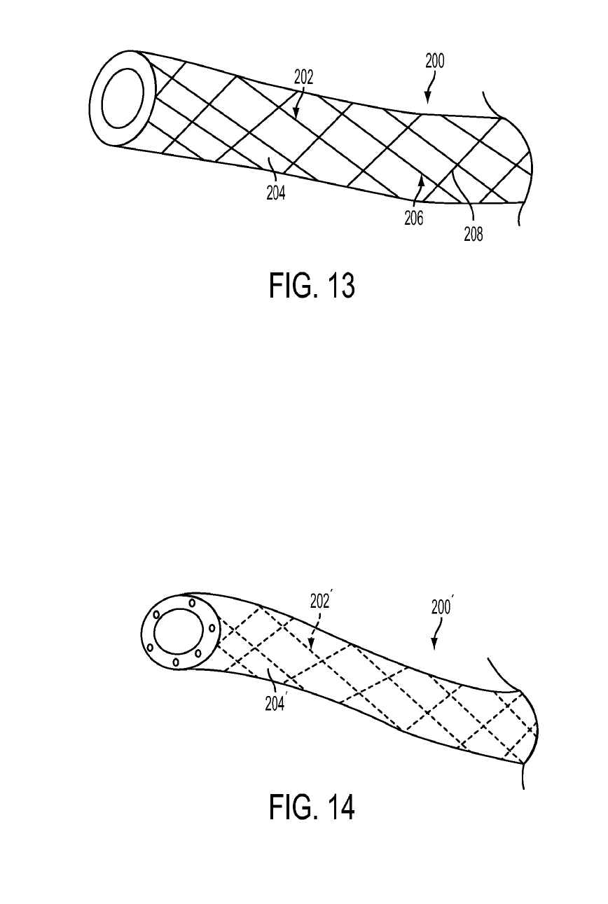

Soft, relatively atraumatic indwelling catheters decrease the rate of adverse events related to the interaction of the catheter with the vascular endothelium. These soft indwelling catheters generally lack sufficient stiffness to be directly placed after needle puncture. FIG. 13 illustrates a catheter 200 formed with a temporary or convertible catheter exoskeleton structure 202 to temporarily increase the stiffness and lubricity of the indwelling catheter 200 during placement over the guidewire, which may be in accordance with the above described embodiments, or not. When this exoskeleton-reinforced indwelling catheter 200 is used as part of the proposed system consisting of a partable needle 100 and the secure guidewire, e.g., any of the guidewires described herein above, the reinforced indwelling catheter 200 can be used instead of a dilator to enlarge the soft tissue tract and the vascular puncture site once the needle 100 has been removed. Of course, a secure guidewire formed with an impediment structure that may be overcome by active intervention does not require use of the partable needle 100 or preloading of the exoskeleton-reinforced catheter.

As depicted in FIG. 13, the catheter 200 includes a lumen-defining portion 204 of a suitable soft, atraumatic material. Formed, as shown in FIG. 13, external to the lumen 204 is the exoskeleton structure 202 consisting of a web 206 of reinforcing members 208. To provide temporary or convertible strength, the exoskeleton structure 202 could consist of a biodegradable co-polymer with a glass transition temperature, T_{g} that approximates body temperature such that the exoskeleton structure 202 is in a stiff glassy state when at room temperature and in a flexible, plastic state when in the vascular lumen for a short period of time. The interested reader is directed to U.S. Pat. Nos. 5,670,161, 6,607,553, and 6,736,842, which disclose a biodegradable stent that uses the temperature properties of a particular co-polymer for deployment, the subject matter of which is incorporated herein by reference in their entireties. Additionally, being of a bio-absorbable or biodegradable substance, the exoskeleton structure 202 breaks down and is removed from the indwelling catheter by hydrolysis or some other means over time. The lumen 204 is bonded or otherwise secured to the exoskeleton structure 202 so that it does not substantially deform independent of or separate from the exoskeleton structure during placement.

In an alternate embodiment depicted in FIG. 14, a catheter, catheter 200′ has the exoskeleton structure 202′ that is incorporated into the lumen 204′ or at least an outer surface portion thereof. In this embodiment, the exoskeleton structure 202′ consists of a co-polymer with a glass transition temperature, Tg that approximates body temperature such that at room temperature or below the exoskeleton structure 202′ is in a stiff glassy state and when placed in the vascular lumen transitions to a soft, flexible plastic state. In use, the catheter 200′ may be cooled below room temperature prior to placement to ensure attainment of the stiff, glassy state prior to placement. Potential polymers or co-polymers that may take advantage of the temperature (Tg) characteristics include polylactide, poly-E-capraoactone or a co-polymer of the two. Potential materials for the exoskeleton more generally that likely could not take advantage of the stated temperature characteristic but that could be partable include—polyurethane, polyethylene, nylon, carbon, Kevlar. These materials could be coated or uncoated to improve lubricity. These coatings could be liquids, various particles including nanoparticles, gels, or solids. The material characteristics could be refined by multiple well-known methods including, but not limited to, the length of the polymer chains; the degree of randomness with which the polymer chains are organized; the degree to which the polymer solutions are “purified” to remove reagents and impurities; the method by which the materials are processed (e.g., temperature, pressure, and draw tension during the extrusion and/or molding processes).

The partability could be imparted during the molding or drawing processes or could be the result of “post-processing” using various well-known techniques such as laser etching and mechanical scoring.

In alternate embodiments, the exoskeleton structure 202′ may be made of a material responsive to an external stimulus to transition from a rigid state to a softened state. The material may be responsive to ultrasonic, magnetic, chemical, laser, heat or other stimulus sources. Additionally, the exoskeleton structure 202′ material may be capable of transitioning from the softened state to a rigid state. The exoskeleton material could have engineered “imperfections” within the base material such as microbubbles that could be activated by the external stimulus thereby causing loss of integrity of the base material. The base material could also have specific energy absorbers such as gold nanoparticles that could increase the absorption of heat or other energy to assist in the degradation or temperature change. In the case of laser energy, these materials could be specific dyes that are included in the base material.

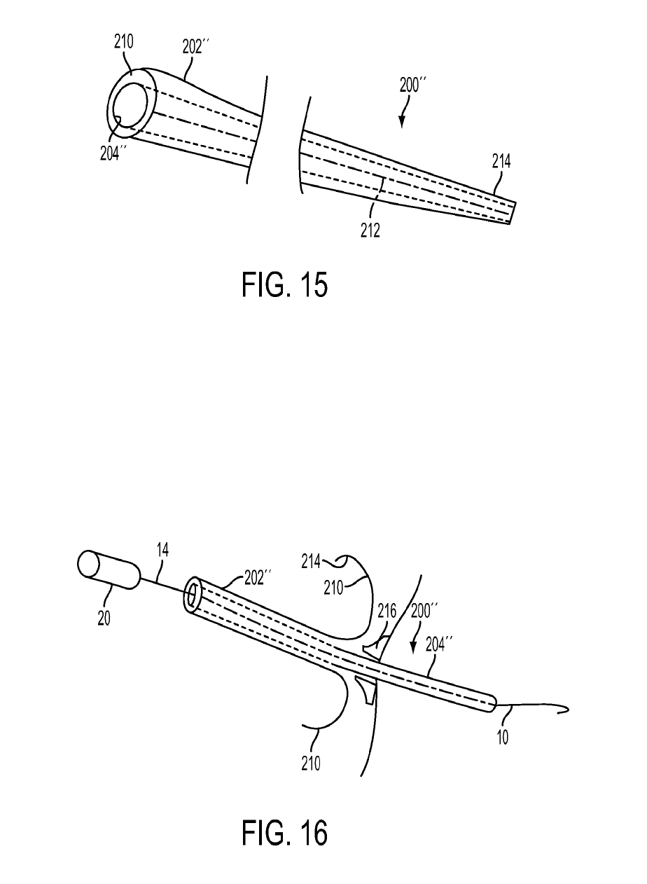

In a further alternative embodiment, as shown in FIG. 15 a catheter 200″ may have an exoskeleton structure 202″ that is a pressure fitted plastic or polymer sheath-like device 210 disposed over the lumen 204″. FIG. 15 is a graphic depiction, and the wall thicknesses are omitted for clarity, although it is clearly understood the lumen 204″ and the exoskeleton structure 202″ each have thickness. This sheath 210 may be scored (212) or prepared to be easily split from the distal tip 214 towards the externalized portion as the indwelling catheter 204″ is advanced over the guidewire 10, as illustrated in FIG. 16. The exoskeleton structure 202″ is retained at the puncture site. The retention of the exoskeleton structure 202″ could be accomplished manually by the operator. To increase ease of use, the exoskeleton structure 202″ could be retained outside the patient by way of a stiff, non-deformable, flanged hub 216 that would exceed the diameter of the skin puncture site. The splitting of the exoskeleton structure 202″ is in the opposite direction as compared with the design of available peel-away sheaths, which generally peel-away from the accessible externalized portion rather than from the tip, which is internalized within the vascular lumen.

The use of an exoskeleton structure, example structures 202, 202′ or 202,″ need not be restricted to the purpose of placing an indwelling catheter. Exoskeleton structures may find utility with the passage of any soft catheter that has greater sliding resistance than desirable during placement, but has the desired mechanical characteristics for its final purpose. The exoskeleton structure would have utility in altering the catheter characteristics during placement, but allow the catheter to retain its desired characteristics to achieve functionality once in place.

### Methods of Preparation and Use

Embodiments of a secure guidewire, partable needle, and catheter exoskeleton structure have been described. These structures may be used individually or in combination, and with traditional guidewire, needle, catheter devices and structures. These structures will find utility in the applications described and may find additional utility for multiple purposes, either alone or in concert. For example, a combination of secure guidewire, partable needle, and exoskeleton structure reinforced indwelling catheter may be used for many types of intra-luminal access. One example procedure, without limitation, is the initial placement of an indwelling central venous catheter. Without the partable needle, a secure guidewire and exoskeleton structure reinforced indwelling catheter may be used or the exchange of a previously placed indwelling central catheter, regardless of whether the originally placed indwelling catheter incorporated an exoskeleton structure or not.

It will be appreciated then that various kits may be constructed that include one, some, or all of the herein described devices and structures along with additional items to accomplish a specific procedure or to be used generally for intra-luminal access. One such kit may include a 1) secure guidewire, and/or 2) partable needle, and/or 3) temporary catheter exoskeleton in accordance with any of the herein described embodiments. A purpose for the kit is to facilitate a procedure or method of either the placement or exchange of an indwelling catheter or similar devices. Such a kit and practiced method has the advantage of decreasing the number of serial steps inherent in the placement or exchange of the indwelling catheter. Additionally, providing and using a secure guidewire or a guidewire fitted with an impediment structure in accordance with any one of the herein described embodiments decreases the likelihood that the operator might inadvertently lose control of the guidewire leading to the guidewire becoming completely within the vascular system and/or the skin tract, but without any externalized portion. Still further, providing and using a secure guidewire or a guidewire fitted with an impediment structure in accordance with any one of the herein described embodiments decreases the likelihood that the operator might inadvertently lose intra-luminal access by inadvertently withdrawing the guidewire, and/or needle, and/or sheath, and/or catheter and thereby leaving no device within the lumen. Kit preparation may be completed and accomplished at the point of care or at the point of manufacture.

While the invention is described in terms of several embodiments of secure guidewires, impediment structures for guidewires, partable needles, and/or exoskeleton reinforced catheters, and corresponding methods of preparation and use, it will be appreciated that the invention is not limited to such structures and methods. The inventive concepts may be employed in connection with any number of devices and methods for providing intra-luminal access.

Additionally, while the structures and methods of the present disclosure are susceptible to various modifications and alternative forms, certain embodiments are shown by way of example in the drawings and the herein described embodiments. It will be understood, however, that this disclosure is not intended to limit the invention to the particular forms described, but to the contrary, the invention is intended to cover all modifications, alternatives, and equivalents defined by the appended claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Unless a claim element is defined by reciting the word “means” and a function without the recital of any structure, it is not intended that the scope of any claim element be interpreted based on the application of 35 U.S.C. §112, sixth paragraph

We claim:

1. A guide wire comprising:
an elongate body portion having a proximal end and a distal end; and
an impediment structure disposed on the guidewire at or adjacent the proximal end.

2. The guidewire of claim 1, wherein the proximal end has a dimension d2 and the impediment structure has a dimension d1 that is larger than d2.

3. The guidewire of claim 1, wherein the impediment structure comprises an enlarged diameter portion of the proximal end.

4. The guidewire of claim 3, wherein the impediment structure comprises a coiled portion of the body portion.

5. The guidewire of claim 3, wherein the impediment structure comprises a deformed portion of the body portion.

6. The guidewire of claim 1, wherein the impediment structure is removeably disposed on the proximal end.

7. The guidewire of claim 1, wherein the impediment structure is permanently secured to the body portion.

8. The guidewire of claim 6, wherein the impediment structure is frictionally secured to the body portion.

9. The guidewire of claim 6, wherein the impediment structure is threaded onto the body portion.

10. The guidewire of claim 1, wherein the impediment structure is bonded to the body portion.

11. The guidewire of claim 1, wherein the impediment structure is deformable and is characterized by an impeding configuration and a release configuration.

12. The guidewire of claim 11, further comprising a member to maintain the impediment structure in the release configuration.

13. The guidewire of claim 10, wherein the impediment structure has a foam or a gel construction.

14. The guidewire of claim 1, wherein the impediment structure has at least one collapsible wing member radially extending from the body portion.

15-36. (canceled)

37. The use of a guidewire in accordance with claim 1 for placing or replacing a catheter having a soft, atraumatic lumen portion and a reinforcing exoskeleton joined with the lumen portion.

38-51. (canceled)
